(19) United States
(12) Patent Application Publication
(10) Pub. No.: US 2007/0020242 A1
(43) Pub. Date: Jan. 25, 2007

(54) Method for transdifferentiation of non-pancreatic stem cells to the pancreatic pathway

(75) Inventor: Vijayakumar Ramiya, Gainesville, FL (US)

Correspondence Address: SWANSON & BRATSCHUN L.L.C., 1745 SHEA CENTER DRIVE, SUITE 330, HIGHLANDS RANCH, CO, 80129, US

(73) Assignee: IXION BIOTECHNOLOGY, INC., Alachua, FL (US)

(21) Appl. No.: 10/550,720

(22) PCT Filed: Mar. 29, 2004
(86) PCT No.: PCT/US04/09624

§ 371 Date: Jun. 13, 2006

Related U.S. Application Data
(60) Provisional application No. 60/458,229, filed on Mar. 27, 2003.

(51) Int. Cl. A61K 35/39; C12N 5/08

(52) U.S. Cl. 424/93.7; 435/366

(57) ABSTRACT

The subject invention relates to a method of transdifferentiating mammalian non-pancreatic stem cells, such a mesenchymal stem cells (MSCs), to enter the pancreatic differentiation pathway. The MSCs are cultured under conditions that permit the expression of pancreatic differentiation markers, and these conditions include use of: (a) culture conditions that promote cell clustering; and/or (b) medium comprising glucagon-like peptide-1 (GLP-1), hepatocyte growth factor (HGF) and/or nicotinamide.

### BACKGROUND OF THE INVENTION

Type 1 diabetes is a polygenic, chronic metabolic disease characterized by the progressive ablation of insulin-producing β cells by autoimmunity. There are nearly 1 million people afflicted by this disease. Regular insulin injections do not maintain blood glucose near normal levels at all times and consequently patients develop secondary complications. While pancreatic and islet transplantations consistently establish euglycemic state and significantly reduce long-term complications, availability of the grafts is severely limited. Xenotransplants, on the other hand, pose a potentially serious threat of xenosis (transfer of animal infections to humans) with attendant regulatory problems and delays. Thus, there is an urgency to develop a β cell/islet replacement therapy for Type 1 diabetic patients that would supply sufficient number of functional human islets on demand. Our laboratory has investigated the potential of adult pancreatic duct-derived islet producing stem cell (IPSC) to provide functional islets for implantation (see WO 95/29988). Unlike IPSCs, the wealth of information on the growth and differentiation of mammalian HSCs and MSCs is vast. Purified HSCs are routinely used in the clinical setting and commercially available for research purposes. Additionally, HSCs and MSCs can be purified from the peripheral blood or bone marrow samples of any individual using commercially available kits. Thus, investigating the feasibility of differentiating human HSCs and MSCs into pancreatic pathway will be of immense importance in curing Type 1 diabetes, and in the realization of autologous stem cell derived insulin producing cells/islets for implantation that will obviate even the usage of immunosuppressive drugs (if encapsulating materials for the protection of cells against autoimmune attack become available). In the subject invention, we have 1) characterized human bone marrow derived CD34⁺ HSCs and MSCs for developmental genes involved in the generation of islets/pancreas following treatments with various factors in vitro, and 2) determined the functional capability of differentiated cells in terms of glucose stimulated insulin secretion in vitro.

Type 1 diabetes is diagnosed by high blood glucose levels resulting from the progressive destruction of insulin producing β cells by autoimmunity. The clinical management of diabetes costs nearly $100 billion each year, and this disease remains the seventh leading cause of death in the USA. Currently there are only a few therapeutic options available for people with Type 1 diabetes, that include administration of extraneous insulin, pancreas and islet cell transplantations (available only for a small selected group of diabetics). Unfortunately, it is impossible to control blood glucose to near-normal levels by regular insulin injections. This failure results in frequent hyperglycemia, which eventually leads to both acute metabolic complications such as diabetic ketoacidosis, hyperosmolar non-ketotic coma and hypoglycemia, and long-term complications such as retinopathy, neuropathy, nephropathy, impotence, heart disease, and foot ulcers. However, a strict control of blood glucose levels with the intensive insulin treatment over a long period of time (years) has been shown to provide impressive primary and secondary prevention of retinopathy, nephropathy, and neuropathy by the Diabetes Control and Complications Trial (DCCT) (1). Despite its beneficial effects, intensive insulin therapy involves drastic changes in patient's lifestyle, increased episodes of hypoglycemia. Hence, it is difficult to motivate patients to undertake these changes. Pancreas and islet transplantations are the only current treatment of Type 1 diabetes that consistently establish an extraneous insulin-independent normaglycemic state, reduce long-term complications, and improve neural and vascular functions (2-8). However, the requirement of immunosuppressive drugs following transplantation can lead to nephrotoxicity, secondary infections, and possibly alteration of glucose homeostasis and cell function (9). Unlike whole pancreas transplantation, islet cell implantation is a simpler surgical procedure, and the most recent islet implantation study has resulted in complete insulin independence in the recipients (8). However, the availability of human pancreata is severely limited, resulting in approximately 3,000 people dying each year while waiting for a graft (10). Despite xenogeneic porcine islets have become an interesting source of islets, they pose more serious threat of xenosis (introduction of animal pathogens into humans) (11). Thus, there is an urgent need for the development of methodologies to create a reliable and safer source of islets, preferably generated in vitro in large numbers to the meet the demand for transplantation. The following narrative on pancreas development provides current status of research on the pancreatic stem cells, and indicates how HSCs/MSCs could augment the chances of success in attempts to cure Type 1 diabetes.

Development of Endocrine Pancreas and Pancreatic Stem Cells

Pancreatic islets of Langerhans possess a remarkable architecture and cellular organization ideal for rapid, yet finely controlled, responses to changes in blood glucose levels. Briefly, the mammalian pancreas controls nutrient resorption and glucose metabolism through its major components, the ductal cells, acinar cells and endocrine cells. Despite all three components of the pancreas differ in functionality, they are all of the same origin, the primitive gut endoderm. During early gestation (28 days in humans) evaginations of the embryonal foregut form the ventral and dorsal buds of the pancreas. The two buds arise opposite to each other while the gut is still surrounded by primitive mesenchyme. After rotation of the stomach and duodenum, the ventral anlage moves around and fuses with the dorsal bud. The ventral bud forms the posterior part of the pancreatic head including the ulcinate process, while the dorsal bud forms the remainder of the organ. In the enlarging epithelial buds, a treelike ductal system develops which eventually gives rise to endocrine and acinar cells (12). It is believed that the “protodifferentiated” epithelial cells which reside in the ducts also share the features of ductal cells (13). These and more recent observations suggest that the pancreatic duct cells harbor the stem cells, which under appropriate stimuli, can give rise to acinar and endocrine cells (14, 15). The pancreatic ductal progenitor stem cells have been shown to express tyrosine hydroxylase (16), glucose transporter (GLUT-2) (17), cytokeratins (18), PDX-1 (19), high-affinity never growth factor TrkA (20), Isl-1 (21), and ngn-3 (22). In the human fetal pancreas, proliferation is mainly found in the ductal cell compartment, followed in frequency by endocrine cells, which are synaptophysin positive but hormone negative, and finally, insulin/glucagon positive cells. In addition, it was noted that all epithelial cells, including endocrine cells, express cytokeratin 19 from 12-16 gestation weeks. This cytokeratin later disappears from the endocrine cells (23).

Finally, in the adults, islet cells have been shown to replicate and respond to stimuli known to initiate neonatal islet cell growth, e.g., glucose, growth hormone, several peptide growth factors, and especially, hepatocyte growth factor (24, 25). These observations suggest that normal β cell growth in the adult can accommodate functional demand. Further, conditions such as obesity and pregnancy result in reversible increase in β cell mass (26-29). Interestingly, there are evidence for a slow turnover of adult cells through stem cell differentiation as demonstrated by a number of experimental models: cellophane wrapping and partial duct obstruction (30), alloxan treatment (31), streptozotocin treatment (32), partial pancreatectomy (33), steroid injection (34), insulin antibody injection (35), copper deficiency condition (36), transgenic interferon-model (37), soybean trypsin inhibitors treatment (38), and specific growth factors treatment (39). Furthermore, in normal conditions, nearly 15% of all 3 cells that are smaller in size compared to mature islet β cells were located in or along ductules in human adult pancreata in immunohistochemical analysis indicating the presence of numerous sites with a potential for β cell neogenesis (40).

One source of implantable pancreatic tissue is adult pancreatic duct-derived IPSCs which differentiate into functional islets (WO 95/29988). Another source is cadaveric pancreatic tissue samples, which however, are very limited since most of the tissues are used for islet transplantation purposes in the clinics. According to the subject invention, another viable source of implantable pancreatic tissue is the transdifferentiated HSCs and MSCs; such stem cells are easily accessible in every individual.

Hematopoietic and Mesenchymal Stem Cells

The mammalian hematopoietic system produces at least eight distinct lineages of mature blood cells in a continuous manner throughout adult life. These lineages include red blood cells, monocytic, granulocytic, basophilic, myeloid cells, the T and B cells and platelets. In this sense, hematopoiesis may resemble other developmental systems such as small intestine, epidermis, hair follicle of the skin, and male germ cells. Other tissue types such as the liver, central nervous system, muscles seem to replenish more slowly, or in response to injury (41). Complex quantitative analyses of HSCs, in some cases, demonstrated that a single transplantable stem cell is both necessary and sufficient to transfer an intact, normal hematopoietic system to a recipient host (42). Although less extensive, other studies have identified candidate stem cells from a number of other tissues (43-47, 14, 15). The proliferation and development of HSCs in vivo is promoted by contact with bone marrow stromal cells and the surrounding extracellular matrix. While there is some ability of soluble cytokines or growth factors to promote survival and proliferation of stem cells and their progeny in the absence of stromal cell matrix, the primitive HSCs can only be maintained, in the long term, when co-cultured with the appropriate stromal cell environment (48). The characterization of CD34 antigen on HSCs, expressed only by 0.5-5% of human bone marrow cells, has enabled the purification of HSCs in commercial quantities. CD34 is not expressed on more mature counterparts (49). Using the long term bone marrow culture system, it has been established that CD34⁺ HSCs can survive in vitro and differentiate when allowed to grow in contact with bone marrow derived stromal cells, which produce a plethora of factors including M-CSF, GM-CSF, G-CSF, IL-1, IL-6, IL-7, TGF-β, LIF, SCF (50).

Both HSCs and MSCs have been suggested to share common bone marrow precursors that express CD34 antigen. Accordingly, CD50− CD34+ cells give rise to MSCs, while CD50+ CD34+ cells give rise to HSCs. Also, circulating cells contained fibroblast-like MSCs (also called fibrocytes) along with HSCs. The MSCs can differentiate into osteocytes, adipocytes and chondrocytes in vitro when appropriate growth factors are provided (56).

Plasticity of Stem Cells

Recent progress in stem cell biology questions the traditional view that cell's fate is sealed when it becomes part of endoderm, mesoderm, or ectoderm, the primary germ layers of the embryo. More specifically, the assumption that the undifferentiated stem cell state, as defined by the ability of stem cell to produce mature cell populations, is limited to the range of cell types characteristic of each individual tissue (by implication, any given somatic stem cell is physically resident within its appropriate tissue) has been called into question (51). For instance, after transplantation into irradiated hosts, genetically labeled neural stem cells were found to produce a variety of blood cell types including myeloid and lymphoid cells as well as early hematopoietic cells (52). The muscle tissue has been shown to contain a population of stem cells with several characteristics of bone marrow-derived HSCs, including high efflux of the fluorescent dye Hoechst 33342 and expression of the stem cell antigens Sca-1 and c-Kit but not CD45. These stem cells have been suggested to be identical to muscle satellite cells, some of which lack myogenic regulators and capable of responding to hematopoietic signals (53). A similar observation has been made by another group who also demonstrated the muscle differentiation potential of a subset of bone marrow-derived stem cells (54). A possibility of HSCs mobilizing during liver failure to increase the regenerative capacity of the liver, though to a lesser extent, has also been documented (55). These observations suggest that the functional plasticity of somatic tissue derived stem cells may be greater than expected. Recently, bone marrow derived stem cells have been shown to populate and differentiate into neural cells (56 & 57). Bone marrow derived MSC has also been demonstrated to express germinal, endodermal and ectodermal genes besides mesodermal genes, suggesting an apparent “multidifferentiated” state. Within a short duration of 5 hours, a relatively simple defined media could elicit neuronal differentiation of 80% of cells (58). Grafting into ischemic brain of rats of human MSCs could ameliorate neurological deficiency by up-regulating host brain plasticity rather than through replacement of neuronal integration into the circuitry of the brain (59). Further, bone marrow cells that co-purify with MSCs could give rise to cells of endodermal and ectodermal phenotypes (60). While reports on the plasticity of stem cells continue to appear, skepticism has been growing on transdifferentiation/dedifferentiation. Most importantly, stem cell fusion resulting in the phenotype of recipient cells has been observed in vitro suggesting it as a potential phenomenon responsible for assumed transdifferentiation in vivo (61 & 62). However, under in vitro conditions described in the present work where bone marrow MSCs are differentiated into pancreatic islet clusters in the absence of any adult mature cell types, ruling out fusion as a mechanism of observed transdifferentiation.

Significantly, there has been no evidence in the literature to date that MSCs can differentiate into endoderm derived pancreatic islets. The subject invention demonstrates that bone marrow derived MSCs can differentiate into islet-like clusters that are capable of secreting insulin in response to glucose.

### WORKING HYPOTHESIS OF THE INVENTION

Based on the current knowledge of the plasticity of stem cells, and without being bound to a particular theory, we hypothesize that the microenvironment of the bone marrow maintains the CD34⁺ HSCs and MSCs in their native pluripotent state and also helps them to commit to distinct hematopoietic lineages (in the case of CD34⁺ stem cells) or adipo, osteo and chondrocytic lineages (in the case of mesenchymal stem cells [63]) (Table 1). However, when these bone marrow derived stem cells are removed from their native bone marrow environment and placed in vitro in the culture plates with defined media, they may commit to non-hematopoietic lineages due to absence of native microenvironment. Recently, using embryonic stem cells, such commitment towards multiple lineages has been documented (64).

TABLE 1

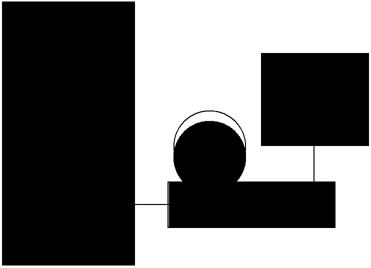

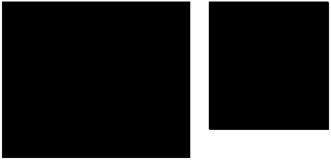

The microenvironment which maintains HSCs and MSCs in their native pluripotent state. Also shown is the list of factors available in that environment that commit these cells into distinct lineages and the phenotype of these stem cells.

The subject invention demonstrates that upon culturing bone marrow derived stem cells in vitro with selected factors, these cells may differentiate into insulin expressing cells or cells expressing transcription factors involved in insulin expression (Table 2) requiring further maturation.

TABLE 2
A select list of transcription factors expressed by hematopoietic
and pancreatic stem cells during development.

Hematopoietic Specific
Transcription Factors
Tal-1, AML-1, CBFb, GATA-1,
GATA-2, GATA-3, PU-1, NF-E2,
PAX-5, SOX-4, EBF, EKLF, IKAROS,
Egr-1
Pancreatic Specific
Transcription Factors
NGN3*, ISL-1*, PDX-1, PAX4, PAX6,
NKX 6.1, NKX 2.2
*Shared by neuronal system during development

### SUMMARY OF THE INVENTION

Type 1 diabetes is suffered by about 1 million people in the USA, and the clinical management of diabetes (Types 1 & 2) costs over $100 billion a year. Regular insulin injections do not maintain blood glucose near normal levels at all times and consequently patients develop secondary complications. While pancreatic and islet transplantations consistently establish euglycemic state and significantly reduce long-term complications, availability of the grafts is severely limited. Xenotransplants, on the other hand, pose a potential threat of xenosis (transfer of animal infections to humans) with attendant regulatory problems and delays. Thus, there is an urgency to develop a β cell/islet replacement therapy for Type 1 diabetic patients that would supply sufficient number of functional human islets on demand.

Mammalian HSCs and MSCs are produced in large quantities throughout life, and are easily obtainable in large quantities. The HSCs could be obtained from peripheral blood or bone marrow samples of an individual. Currently, these HSCs are used in the clinical setting and commercial quantities of purified HSCs and MSCs are available for research purposes. Thus, the transdifferentiation of HSCs and MSCs into pancreatic pathway can be of immense importance in curing Type 1 diabetes. The subject invention demonstrates the transdifferentiation of MSCs into glucose-responsive insulin secreting islet-like clusters. Further, bone marrow MSC derived islet clusters have and continue to be characterized, differentiation procedures have been and can continue to be fine-tuned, and in vivo functionality can be established in the mouse model.

The subject invention provides a new hope for an insulin-free life for every single diabetic (both insulin-dependent Type 1 and Type 2 patients) and it will require only their peripheral blood or bone marrow samples to achieve it. There will not be any need for immunosuppressive drugs when appropriate encapsulation material is used for protection of insulin producing cells from autoimmune attack in Type 1 diabetics. The commercial value of this approach is immense both in terms of transplantable products and services that could be delivered to clinics. Further, HSCs and MSCs, at different stages of differentiation, could be used for drug discovery research.

The subject invention provides a method for transdifferentiation of mammalian non-pancreatic stem cells to the pancreatic differentiation pathway such that the cells express pancreatic differentiation markers. The culture conditions for transdifferentiation include conditions that promote cell clustering; and/or a medium that comprises an added factor that is at least one of glucagon-like peptide-1 (GLP-1), hepatocyte growth factor (HGF) and/or nicotinamide.

The pancreatic markers that can be expressed in the transfected cells include any of Pdx-1, Isl-1, Pax-4, Pax-6, Glut-2, glucagon, somatostatin, pancreatic peptide (PP) and insulin. Preferably, the transdifferentiated cells respond to contact with glucose by secretion of insulin. The culture conditions that promote cell clustering include low binding tissue culture plates (which typically are not coated), extracellular matrix, and/or other methods known in the art.

The transdifferentiation medium comprises one or more of the following components: Dulbecco's Minimal Essential Medium (DMEM) with high glucose and sodium pyruvate; bovine serum albumin (BSA); 2-mercaptoethanol; fetal calf serum (FCS); penicillin and streptomycin (Pen-Strep); insulin, transferrin and selenium (ITS); and Fungizone®. The culture can comprise further added factors selected from the group consisting of stem cell factor (SCF), activin A, betacellulin, epidermal growth factor (EGF), keratinocyte growth factor (KGF), prolactin (PRL), nerve growth factor (NGF), transforming growth factor-α (TGF-α), gastrin, extendin-4, basic fibroblast growth factor (bFGF), and conditioned medium of a cell line selected from the group consisting of Capan-1, ARIP and AR42J. Preferably, the added factors comprise GLP-1, HGF and nicotinamide. It is also preferred that GLP-1 have a concentration of about 100 nM, HGF a concentration of about 20 ng/ml, and nicotinamide a concentration of about 10 nM.

In one embodiment, the non-pancreatic stem cell is a mesenchymal stem cell (MSC). The MSC is preferably CD105+, CD166+, CD29+ and CD44+. It is also preferred, though not necessary, that the stem cells are human.

The subject invention also includes a method of producing an endocrine hormone comprising the above-described transdifferentiation method, and further comprising the step of continuing to culture said transdifferentiated cells in a medium (such as that described in Table 3), whereby an endocrine hormone may be produced.

The subject invention also includes a method of treating a mammal with a pancreatic disorder by culturing non-pancreatic stem cells as described herein, whereby the stem cells transdifferentiate to the pancreatic differentiation pathway; and using a product of the culture step to treat the mammal. The pancreatic disorder can be an insulin-requiring disorder such as insulin-dependent diabetes (IDD). The product of the culture method can be an endocrine hormone which is then administered to the mammal. Alternatively, the product of the culture method is transdifferentiated cells, which are then implanted in the mammal. Implantation may be in pancreatic, kidney or liver tissue, or in a subcutaneous pocket. Preferably, the implanted, transdifferentiated cells are transdifferentiated MSCs. The implanted, transdifferentiated cells may be allogeneic or autologous. The implanted transdifferentiated cells can be encapsulated in an endocrine hormone permeable capsule.

In one embodiment, the transdifferentiated cells are modified to substantially reduce expression of an antigen selected from the group consisting of insulin dependent diabetes associated autoantigens, GAD, 64 kD islet cell surface antigen and human leukocyte antigens, whereby the modified transdifferentiated cells do not substantially express said antigen.

The subject invention also includes the transdifferentiated cell produced by the transdifferentiation methods described herein, and in particular the transdifferentiated MSC produced by said methods. Such cell preferably expressed mRNA for Isl-1, Pax-6, PP, insulin, glucagon and somatostatin. The subject invention also includes a therapeutic composition comprising a transdifferentiated MSC encapsulated in an endocrine hormone permeable capsule.

As described herein, the subject invention demonstrates the feasibility of differentiating human bone marrow derived MSCs into pancreatic islet-like clusters that are capable of secreting insulin in response to glucose in vitro. Less success has been achieved at this time with transdifferentiation of HSCs to the pancreatic pathway.

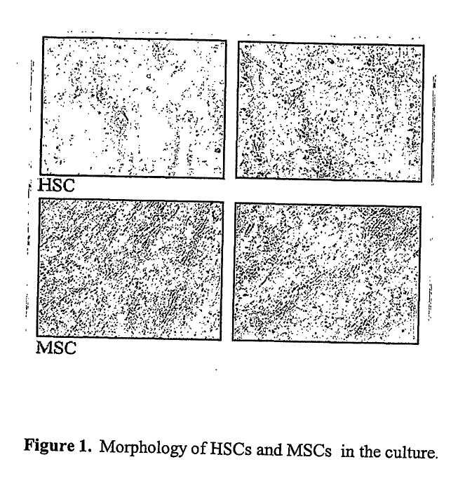

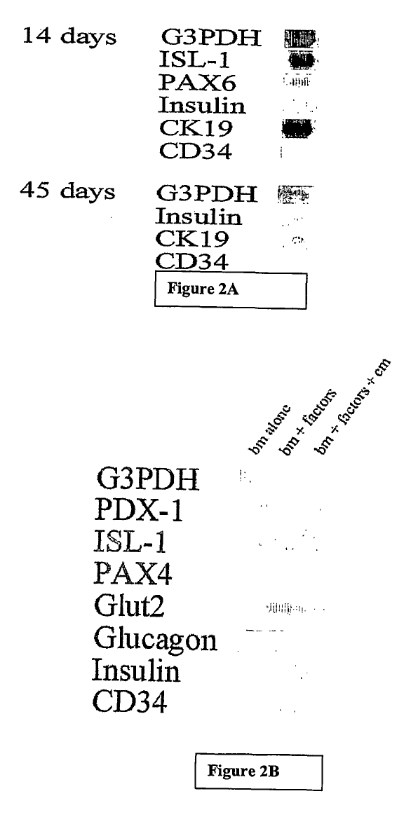

By culturing human bone marrow derived stem cells in vitro, we demonstrated that they can enter into non-conventional differentiation pathways, a process called “transdifferentiation”. Purified human bone marrow stem cells were purchased from Clonetics (BioWhittaker, Walkersville, Md.). Purity of the cell populations exceeded 95% according to the supplier. Cryopreserved cells were thawed and cultured overnight with HSC specific growth media and MSC specific growth media (Clonetics) as suggested by the manufacturer (FIG. 1) overnight prior to culturing in our medium conditions. While HSCs are round cells, MSCs exhibit typical fibroblast-like spindle shaped cells. Our original observation with a mixture of CD34+HSCs (CD34+) and MSCs (CD34−, CD45−, CD14−, CD44+, CD29+, SH2+, SH3+) indicated that such differentiation into endocrine pancreatic pathway was feasible (see WO 02/079457). The composition of the media used is given in Table 3. This medium combination was called “combination 1”. Briefly, the media contained several selected factors. Upon culturing the mixed stem cells in this medium, insulin expression could be demonstrated around 6 weeks later (FIG. 2A). When pure MSC population was cultured in the same medium (combination 1), after 3 weeks in culture, mRNA expression for Pdx-1, Isl-1, glucagon, and Glut-2 were observed (FIG. 2B). However, no insulin transcription was evident. Thus, we then extended the observation with 4 additional combinations of media (variations to the original medium, combination 1). Table 4 shows the variations designed. The basal media component was not changed except for fetal calf serum (FCS), which was brought down to 2%. All other variations were introduced in the addition of factors. During evaluation of combinations 2-4, doses of factors were kept constant to avoid another variable in the experimental design. The “best” dose was selected from doses reported in the literature for each component to be non-toxic, or provided by the manufacturer to have biological activity in their standard assays.

TABLE 3
Basal medium components and factors/reagents used for
differentiation of HSCs and MSCs

Basal Medium Components (combination 1)
DMEM with high glucose and sodium pyruvate (Dulbecco's
Minimum Essential Medium, Gibco-BRL)
0.1% BSA
0.1 mM 2-Mercaptoethanol
15% FCS (HyClone)
1x ITS (insulin, transferrin and selenium) (Gibco-BRL)
Penicillin 100 IU/ml and streptomycin 100 μg/ml
Fungizone ® 1 μg/ml (Gibco-BRL)
Factors Added to Basal Medium
GLP-1 (glucagon-like peptide) 10 nM
Activin A 1 nM
Betacellulin 5 nM
HGF (hepatocyte growth factor) 20 ng/ml
EGF (epidermal growth factor) 20 ng/ml
KGF (keratinocyte growth factor) 20 ng/ml
NGF (nerve growth factor) 3 ng/ml
TGF-alpha (transforming growth factor) 10 ng/ml
Conditioned medium (of Capan-1, ARIP and AR42J cell lines) 10%
Nicotinamide (10 nM)
Extendin-4 (100 nM)
bFGF (5 ng/ml)

TABLE 4
Media Combinations used. Media combinations designed from
original media combination 1. As indicated, the variations were
only with the set of factors introduced. The dosage of the factors
were kept constant to avoid additional variability.

| | |
|---|---|
| Media | Basal medium components (2% FCS) + Factors: activin |
| combination 2 | (1 nM), beta cellulin (5 nM), HGF (20 ng/ml), |
| | EGF(25 ng/ml) |
| Media | Basal medium components (2% FCS) + Factors: KGF |
| combination 3 | (20 ng/ml), NGF (10 ng/ml), TGF-alpha (10 ng/ml) |
| Media | Basal medium components (2% FCS) + Factors: GLP-1 |
| combination 4 | (100 nM), HGF (20 ng/ml), nicotinamide (10 nM) |
| Media | Basal medium components (2% FCS) + Factors: |
| combination 5 | Extendin-4 (100 nM), FGF-basic (5 ng/ml), nicotinamide |
| | (10 nM), HGF (20 ng/ml). |

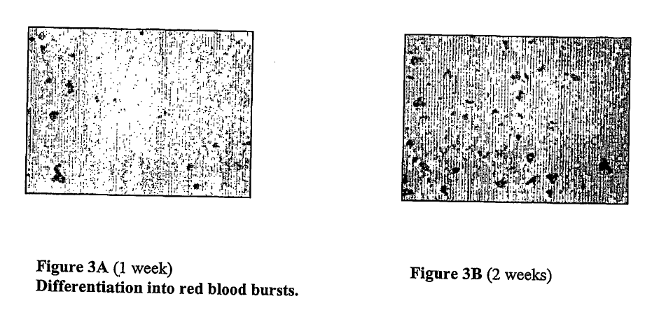

In all media combinations tested, human bone marrow derived HSCs differentiated mostly into erythroid bursts starting within 2 weeks in culture (FIG. 3). There was no expression of endocrine related genes such as Pdx-1, Isl-1, beta2/NeuroD, glut-2 or endocrine hormone transcripts (data not shown). After 3 weeks no cells remained viable. These observations convinced us to restrict further investigations to MSCs.

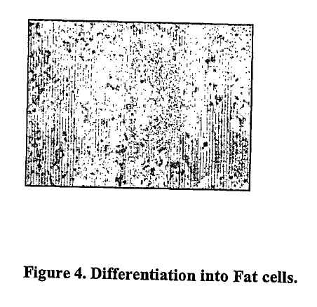

Cultured MSCs which adhere to the tissue culture plates appear morphologically like fibroblasts. They are maintained for one or two days in MSCGM™ medium (Clonetics/BioWhittaker, Walkersville, Md.) which enables their growth without inducing differentiation. Upon treatment with adipogenic differentiation medium (Clonetics/BioWhittaker), in 2 weeks culture, about 70% of the cells become fat cells. This confirmed the retention of differential potential of MSCs in our hands (FIG. 4).

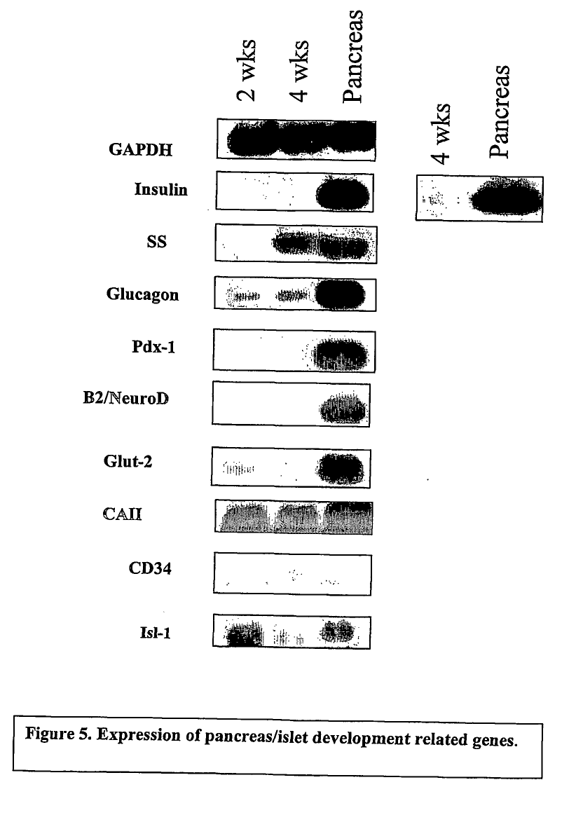

MSCs were cultured at 0.5×10⁶ cells per T25 tissue culture flasks. Cells were cultured in media combinations shown in Table 4. Cultures were continued for 1, 2, and 4 weeks. At the end of each culture period, RT-PCR analysis was performed to detect any expression of genes relevant to pancreatic/islet developmental pathway. While media combinations 1, 2, 3 (see Table 4) were not able to induce any pancreas/islet relevant gene expression, interestingly, media combination 4 that contained nicotinamide (10 mM), glucagon-like peptide (GLP-1; 100 nm), and hepatocyte growth factor (HGF; 20 ng/ml) induced pancreatic genes of interest (FIG. 5).

Briefly, MSCs cultured with media combination 4 expressed glucagons and Somatostatin (SS). Glut-2 and Isl-1 are also evident. Interestingly CD34 mRNAs were also expressed despite the fact that purified MSCs are negative for CD34. However, our previous experience with a mouse adult pancreatic progenitor cell line where CD34 mRNA expression did not translate into surface expression cautions any speculation until a FACS analysis is conducted. At 4 weeks, there is a basal level of expression of insulin. This low level transcription was reproducible as shown in the additional inset. Equally interesting was the absence of detectable level of Pdx-1 expression which is considered a master switch in pancreas/islet development and function. Insulin expression without detectable Pdx-1 has been noted in mouse embryonic stem cells. There was no expression of insulin, SS, glucagons in MSCs treated with only MSCGM™ medium, or media combination 4 without factors (data not shown). Carbonic anhydrase II was expressed by undifferentiated MSCs. Despite the ability of media combination 4 to induce expression of pancreas development related genes, it is believed that the frequency of conversion of cultured MSCs into pancreatic pathway under these conditions is low. MSCs treated for 4 weeks in media combination 4, when subjected to adipocyte differentiation conditions, fat cells were formed as detected by Oil Red O staining (data not shown). This perhaps indicates that under these conditions (combination 4, adherent culture plates) the differentiation towards pancreatic pathway is of low frequency and may be reversible. Similar observation has been made in neural cell differentiation (58). During all these culture conditions MSCs remain attached to the plastic tissue culture flasks.

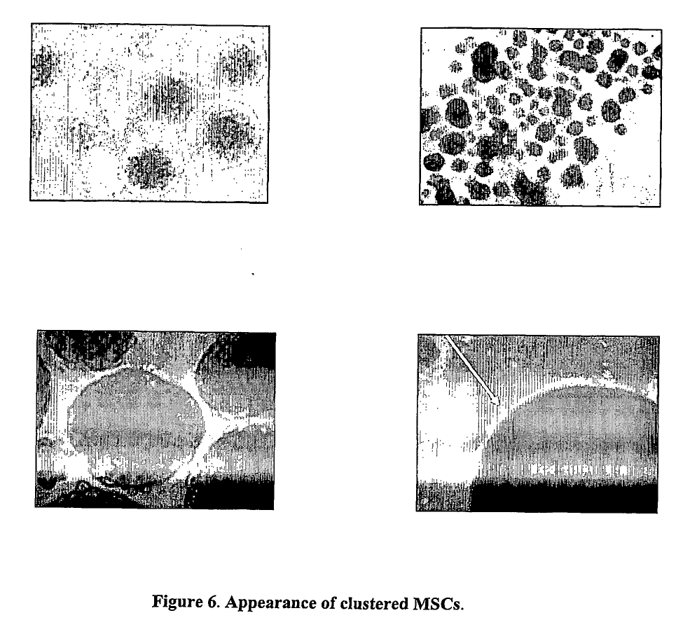

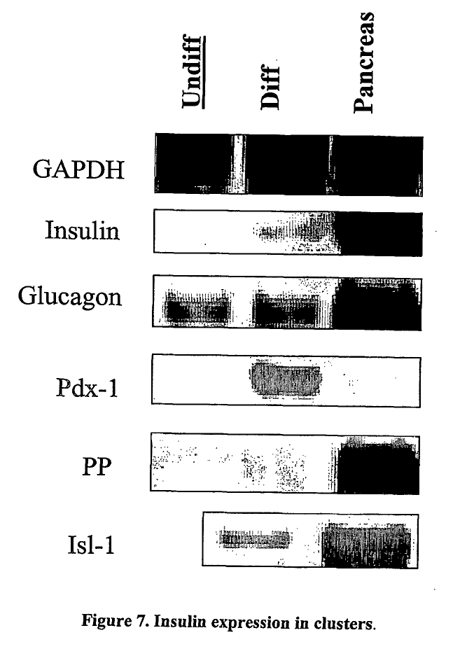

Since clustering or aggregation is considered to be a signal for differentiation (65), we induced clustering of MSCs as a means of encouraging transdifferentiation. Upon arrival, human bone marrow MSCs were plated in low binding tissue culture plates (e.g., from Corning) at 0.5×10⁶ cells in media combination 4. Cells grew as three-dimensional clusters (FIG. 6). Control cells were treated with media combination 4 that did not receive factors (nicotinamide, GLP-1 and Hgf). Cells were cultured for 1 week with fresh media change every 2 days. GLP was added every 24 hrs. At the end of 7 day culture period, cell clusters (50-500 μm in diameter similar to islets, including an outer capsule at arrow) were subjected to RT-PCR analysis. As shown in FIG. 7, clusters cultured for 1 week in media combination 4 (indicated as Diff), expressed insulin, Pdx-1, and a low level of pancreatic polypeptide (PP). Clusters cultured in media without growth factors are indicated as Undiff. Human pancreatic cDNA used a positive control.

The level of insulin expression in 1 week culture was substantial and this observation is significant since adherent MSCs grown in the media combination do not express insulin until 4 weeks and at much lower levels. No comparison was made with Isl-1 levels due to unavailability of data for undifferentiated clusters. More studies are ongoing, and media combination 5 is being tested.

To assess the degree of transdifferentiation towards islet cell functionality, the following assays or analyses were and/or should be conducted: transcriptional expression of insulin, translation of insulin, capability to secrete insulin, the glucose-responsiveness in insulin secretion pattern, and the quantity of insulin secreted in comparison to native pancreatic islets. As described herein, insulin secretion was evaluated in a “shotgun” approach.

Briefly, after 1, 2, and 4 weeks of culturing adherent MSCs in media combination 4 that induced low level expression of insulin at transcriptional level (FIG. 5), media was changed to glucose free, insulin free RPMI 1640, washed 6 times and incubated with the same medium for overnight to “rest” them. Then fresh RPMI 1640 was added with 0, 5.5 and 17.5 mM glucose in static incubation conditions for 16 hrs. Supernatant was cleared of cells, and used in human insulin ELISA kits with appropriate positive insulin controls. There was no detectable insulin in these cultures (data not shown).

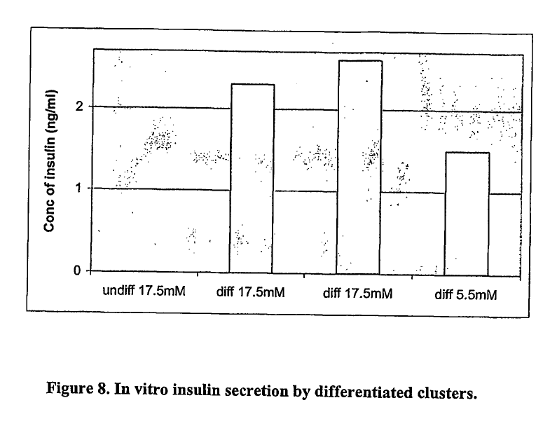

We then did similar study with clustered cells cultured for a week in media combination 4. The results are preliminary but astounding. As shown in FIG. 8, clusters cultured in media without growth factors of combination 4 (Undiff) did not secrete insulin upon stimulation with 17.5 mM glucose, while clusters cultured in media combination 4 (Diff) secreted insulin both at 5.5 mM and at 17.5 mM. With 17.5 mM, the level of insulin secreted was 1.6 times higher than that with 5.5 mM glucose (2.3, 2.6 ng/ml vs. 1.5 ng/ml). Each culture group contained ˜50 clusters of 150-400 uM diameter and were stimulated for 16 hours.

We have demonstrated that human bone marrow derived MSCs can be differentiated into pancreatic differentiation pathway, under adherent cell conditions, to a limited degree. While insulin and pancreas development related genes could be induced, thus far, we have not succeeded in our attempts detect insulin secretion.

Under cell clustering conditions, the differentiation into pancreatic/islet pathway may be more efficient in that substantial level of insulin and Pdx-1 expression could be seen as early as 1 week after the initiation of cultures in differentiating media combination 4. Further, preliminary evidence suggest impressive insulin release which is not so common in stem cell derived islet cell studies.

### EXAMPLES

In above-described experiments, we determined that clustering of human bone marrow derived MSCs and culturing of clustered cells with defined set of factors (combination 4) resulted in a successful differentiation of clusters into glucose-responsive insulin secreting cells. However, HSCs did not transdifferentiate, but instead became bursts of red blood cells in the culture conditions tested. Thus, subsequent experiments focused on characterizing MSCs in terms of their clustering, differentiation, in vito functional physiology and in vivo functionality.

### Example 1

### Characterization of Human Bone Marrow Derived MSCs in Terms of Their Abilities to Form Islet-Like Clusters and Their Differentiation Potential Under Such Circumstances In Vitro

Human bone marrow MSCs are purchased from Clonetics (BioWhittaker, Walkersville, Md.) for all studies. Since media combination 4 has proven to be effective, and media combination 5 is being tested, all 5 combinations of media (Table 4) are used. Briefly, 0.5×10⁶ MSCs are plated in low-binding tissue culture plates (6 well clusters). Cultures are for 1, 2 and 4 weeks with media change every 2-3 days. At the end of time points cells are subjected RT-PCR for pancreatic differentiation pathway as described below. Clusters are also fixed in 4% formalin, immobilized in 4% low-melt agarose for tissue sectioning and immunohistochemistry. Once a more efficient differentiation medium condition is found (currently, media combination 4 appears good; but other combinations have not been tried with clustered cells), we can use at least 5 different doses of each growth factor in a given media combination to “fine tune” differentiation capabilities. We can keep the current dose of factors in the middle and go either way by two doses. Following fine tuning of differentiation media, we can initiate the clustering procedure using Extracellular matrix proteins (ECM gel; Sigma chemicals, St Louis, Mo.). The appropriate procedures are described below.

RT-PCR/Southern Blotting Analyses:

Total RNA is prepared using RNAqueous™-4PCR kit (Ambion Inc., Austin, Tex.). All primers have been designed based on sequences of open-reading frames obtained from GENBANK. One μg of RNA_will be used for reverse transcription, and 1 μg of cDNA will be used for PCR amplification. PCR primers will be synthesized by Life Technologies, Inc. (Gaithersburg, Md.) for endocrine products (insulin, glucagon, somatostatin, and pancreatic polypeptide), exocrine products (amylase and elastase), mesenchymal marker (vimentin), enzymes (hexokinase and glucokinase), receptors (c-Met and GLUT-2), transcription factors (PDX-1, ISL-1, Beta2/Neuro-D, Pax-4, Pax-6 HNF-1, ngn-3, NKX6.1, NKX 2.2, Reg, and PtflP48) and other general markers (CD34, tyrosine hydroxylase, β-galactosidase, HGF, EGF, Albumin, GAD-65, IGF-I, and IGF-II). PCR products can be size separated by gel electrophoresis in 1.5% gel agarose and transferred to nylon membranes by vacuum blotting and UV-crosslinking. The specificity of the PCR amplifications can be predetermined by internal sequence specific probes which have been previously digoxigenin-labeled. Digoxigenin-labeling of an Oligo probe for Southern blotting is carried out using Dig Oligonucleotide Tailing Kit (Roche Molecular Biochemicals, Indianapolis, Ind.) and following the manufacture's protocol. Southern blot is carried out following the PCR reaction using the standard protocol established in the laboratory and known in the art.

Immunohistological Staining and Immunofluorescence Assays:

Cells can be fixed in 4% formalin, and immobilized in 4% low melt agarose for tissue sectioning. Sections can be stained using antibodies to insulin, glucagon, somatostatin, pancreatic polypeptide, amylase, vimentin, tyrosine hydroxylase, carbonic anyhydrase II, β-galactosidase, hexokinase and PDX-1 (generated by Genemed Synthesis, Inc. San Francisco, Calif.). Biotinylated secondary antibodies conjugated with alkaline phosphatase or horseradish peroxidase and alkaline phosphatase and peroxidase conjugated streptavidin can be used to bind to primary antibody. Antibodies will then be visualized using DAB, AEC, Fast Red, or BCIP/NBT. This system can also be used for double staining which can visualize multi-antigen expression. Immunofluorescent staining can also be used for surface staining. This can be done using cells in suspension. These cells can be blocked and then incubated in primary antibody (as mentioned above). Cells can be washed and centrifuged to remove remaining antibody. Secondary antibody conjugated to Alexa 488, Alexa 568, or Alexa 350 (Molecular Probes, Inc., Eugene, Oreg.) can be used to visualize antigens present.

Insulin and Glucagon Assays:

At the end of differentiation procedures, clusters can be washed and seeded in 6 well (low binding) plates, 50 clusters per group. Clusters can be thoroughly washed (6 times) and rested in glucose free, insulin free RPMI medium containing 2% FCS for 16 hrs. After fresh media of the same kind can be added with varying glucose levels (0, 5.5, 11.0, and 17.5 mM glucose) for 18 hrs in a static incubation procedure. If insulin secretion is demonstrated, then similar assays can be planned for 3 hrs. The cell free supernatant can be stored in −70° C. until used in assays. The cells can then be lysed using lysis buffer to extract intracellular insulin content. Quantitation of insulin, both in the supernatant and intracellular content, can be assayed using commercially available Ultrasensitive ELISA kits (Mercodia, Uppsala, Sweden). Glucagon secretion/intracellular content can also be determined using a glucagon ELISA developed in our laboratory. Concentrations of hormone can be normalized with DNA content.

### Example 2

### Characterization of MSC Derived Islet Clusters in Terms of Their Physiological Functions In Vitro

Preferably, only the best differentiation combination media is used for the physiological studies. MSC derived islet clusters are used in optimized standard operating procedures (SOPs) to determine in vitro glucose-responsive insulin secretion at different time points in static incubation cultures as well as glucagon production to evaluate the reproducibility, stability of differentiated phenotype, metabolic features (e.g., glucokinase/hexokinase ratio), sensitivity to counter-regulatory hormones (i.e., somatostatin and glucagon), phosphorylation pattern following glucose binding to its receptor, and potential for “reverse” differentiation into mesenchymal derivatives (e.g., fat cells) following appropriate treatment.

Insulin and glucagons testing procedures have been already described above. The differentiated clusters can be maintained in differentiating media as well as in non-differentiating media conditions for 1, 2 and 3 weeks to test the stability of the differentiated phenotype (insulin and glucagons secretion). Simultaneously “reverse differentiation” potential can be determined by treating differentiated clusters (capable of insulin secretion) with adipogenic differentiation medium. A range of doses of glucagons, somatostatin will be used in insulin secretion assays to investigate the sensitivity of differentiated islet clusters to counter-regulatory hormones, and hence a possible insight of these clusters behavior in vivo. Hexokinase and glucokinase ratios can be monitored by RT-PCR to observe if any changes in the ratio during extended culturing period i.e., 1, 2, and 3 weeks in culture conditions. We have primers and probes already routinely used in our labs.

Glucose Induced Substrate Phosphorylation Assay:

The purpose of this experiment is to compare and contrast downstream signaling events following glucose binding to Glut-2 receptor. Differentiated insulin producing cells are used to determine the glucose stimulated phosphorylation of downstream substrates as a way to investigate the physiological similarities with and variations from the pancreatic islets. Half a million differentiated cells (capable of insulin production) are homogenized in extraction buffer (20 mmol/1 K₂HPO₄, pH 7.5, 5 mmol/1 DTT, 1 mmol/EDTA, protease cocktail and 110 mmol/1 KCl) following stimulation with 17.5 mM glucose for 0, 5, and 15 min. The homogenate will be separated on 10% SDS-PAGE (BioRad, Hercules, Calif.) and the phosphorylated protein substrates will be detected using phosphotyrosine antibody in western blot technique. For comparison, we can use human pancreatic islets (100 islets per time point) to determine phosphorylated substrate pattern following glucose stimulation.

Western Blot Technique:

Following gel transfer (Mini Trans-Blot Cell, Bio-Rad, Hercules, Calif.) to nitrocellulose membrane (Trans-Blot Transfer Medium, Supported 0.45 um Nitrocellulose Membrane, Bio-Rad, Hercules, Calif.) in transfer buffer (25 mM Tris, 192 mM glycine and 20% v/v methanol: pH 8.3), the membrane is washed with Tris-buffered saline (TBS) for 15 min, blocked in TBS containing 5% nonfat dry milk (Price Chopper supermarket) for 1 hr at RT, washed 3 times in TTBS (20 mM Tris, 500 mM NaCl, 0.05% Tween-20; pH7.5), and blotted using rabbit anti-phosphotyrosine antibody at 4° C. overnight at manufacturer's recommended dilution. The membrane is then washed 3 times in TBS, incubated in the corresponding secondary antibodies linked with alkaline phosphatase and diluted 1:1,000 in TTBS for 1 hour at RT, and developed in 10 ml of carbonate buffer (0.1 M NaHCO₃, 1 mM MgCl₂, pH 9.8) containing 60 μl of nitro blue tetrazolium (NBT) solution (dissolve 50 mg of NBT in 0.7 ml of N,N-Dimethylformamide (DMF) with 0.3 ml dH₂O), and 60 μl of 5-bromo-4-chloro-3-indolyl phosphate (BCIP) solution (dissolve 50 mg of BCIP in 1 ml of 100% DMF) until appropriate color obtained. The membrane is then washed in ddH₂O to stop the reaction.

### Example 3

### Characterization of In Vivo Functionality of MSC Derived Islet Clusters in NOD-Scid, Nod Mice and In Vivo Migration Pattern of Implanted Cells

Human MSC derived differentiated clusters can be implanted in the immune-deficient female NOD-Scid mice, followed by implantation in autoimmune NOD. Diabetes can be induced in Scid mice using 160 mg/kg body wt streptozotocin (STZ). This is a routine procedure done in our labs. There can be 1 group of mice with human islets (n=6; 600 islets per animal) and 4 groups of mice with differentiated clusters (n-6 per group; 4 doses of clusters-300, 600, 1200, 2400; therefore totally 24 mice). Implant site is preferably the kidney capsule. Similar studies can be done with diabetes induced female NOD mice (therefore a total of 24 mice). NOD mice can receive immunosuppressive drugs sirolimus (Wyeth-Ayerst) (0.1 mg/kg) and tacrolimus (Fujisawa Canada) (1.0 mg/kg) starting 3 days prior to implantation. After determining the best dose for reversal of diabetes, that dose alone is used in the NOD female diabetic mouse for intraportal implantation (therefore total mice=6) to mimic current human Edmonton protocol. Our lab has extensive experience in intraportal procedures in the mouse. Human pancreatic islets are used in the same fashion for a positive control (n=6). The animals are followed for glucose monitoring every other day, and weekly peripheral insulin levels (using human c-Peptide insulin Kits; Mercodia, Uppsala, Sweden). Monitoring is carried out for 2 months post implantation. At the end of study, implants sites are used to determine morphology of implants, hormone expression and infiltration of immune cells by immunohistochemistry.

For migration studies we can use vital stain CFSD SE to stain living differentiated clusters and will follow their retention or migration from implanted intraportal site. Only one dose that reversed diabetes upon intraportal implantation is used in female non-diabetes prone Balb/c mice. Various tissues will be collected from 3 hr, and 1, 3, 6 and 10 day time points. Each time point will have 4 mice (therefore a total of 20 mice).

All references cited herein are incorporated in their entirety by reference.

### LIST OF REFERENCES CITED

1. The DCCT research group. N Engl J Med 329, 977 (1993).
2. D. E. R. Sutherland, Clin Diabetes 15, 152 (1997).
3. J. S. Najarian, A. C. Gruessner, M. B. Drangsteveit, R. W. G. Gruessner, F. C. Goetz, D.E.R. Insulin independence for more than 10 years in 32 pancreas transplant recipients from a historical era. Southerland, Transplant Proc 30, 279 (1998).
4. P. Fioretto, M. W. Steffes, D. E. Sutherland, F. C. Goetz, M. Maur, Reversal of lesions of diabetic nephropathy after pancreas transplantation. N Engl J Med 339, 69-75 (1998).
5. T. Nymann, D. K. Hathaway, T. E. Bertorini, M. H. Shokouh-Amiri, A. O. Gaber, Studies of the impact of pancreas-kidney and kidney transplantation on peripheral nerve conduction in diabetic patients. Transplant Proc 30, 323-324 (1998).
6. T. Hayashi, M. Nozawa, K. Soyimiya, H. Toko, M. Nakao, M. Okabe, F. Terasaki, Y. Kitaura, K. Kawamura, Transplant Proc 30, 335-338 (1998).
7. B. L. Johansson, A. Kernell, S. Sjoberg, J. Wahren, Influence of combined C-peptide and insulin administration on renal function and metabolic control in diabetes type 1. Influence of combined C-peptide and insulin administration on renal function and metabolic control in diabetes type 1. J Clin Endocrinol Metab 77, 976-981 (1993).
8. A. M. Shapiro, J. R. Lakey, E. A. Ryan, G. S. Korbutt, E. Tote, G. L. Warnock, N. M. Kneteman, R. V. Rajotte. Islet transplantation in seven patients with type 1 diabetes mellitus using a glucocorticoid-free immunosuppressive regimen. N Eng J Med 343, 230-238 (2000).
9. R. Gunnarsson, G. Klintmalm, G. Lundgren, H. Wilczek, J. Ostman, C. G. Groth. Lancet 2, Deterioration in glucose metabolism in pancreatic transplant recipients given cyclosporin. 571-572 (1983).
10. Fact sheet on Xenotransplantation. FDA Backgrounder Sep. 20 (1996).
11. F. H. Bach, J. A. Fishman, N. Daniels, J. Proimos, B. Anderson, C. B. Carpenter, L. Forrow, S. C. Robson, H. V. Fineberg, Uncertainty in xenotransplantation: individual benefit versus collective risk. Nature Med 4, 141-144 (1998).
12. J. Peters, A. Jurgensen, G. Kloppel, Ontogeny, differentiation and growth of the endocrine pancreas. Virchows Arch 436, 527-538 (2000).
13. R. Pictet, W. J. Rutter, Development of the embroyonic endocrine pancreas. In: Geiger S R (ed) Handbook of physiology, sect 7: Endocrinology. Waverley press, Baltimore, PP 25-66 (1972).
14. V. K. Ramiya, M. Marais, K. E. Arfors, D. E. Schatz, A. B. Peck, J. G. Cornelius, Reversal of insulin-dependent diabetes using islets generated in vitro from pancreatic stem cells. Nat Med 6, 278-282 (2000).
15. S. Bonner-Weir, M. Taneja, G. C. Weir, K. Tatarkiewicz, K. H. Song, A. Sharma, J. J. O'Neil, In vitro cultivation of human islets from expanded ductal tissue. Proc Natl Acad Sci USA 97, 7999-8004 (2000).
16. G. Teitelman, S. Alpert, J. M. Polak, A. Martinez, D. Hanahan, Precursor cells of mouse endocrine pancreas coexpress insulin, glucagon and the neuronal proteins tyrosine hydroxylase and neuropeptide Y, but not pancreatic polypeptide. Development 118, 1031-1039 (1993).
17. K. Pang, C. Mukanowashuro, G. C. Wang, Proc Natl Acad Sci USA 91, 9559-9563 (1994).
18. L. Bouwens, R. N. Wang, E. De Blay, D. G. Pipeleers, O. Kloppel, Cytokeratins as markers of ductal cell differentiation and islet neogenesis in the neonatal rat pancreas. Diabetes 4, 1279-1283 (1994).
19. J. Jonsson, L. Carlsson, T. Edlund, H. Edlund. Insulin-promoter-factor 1 is required for pancreas development in mice. Nature 371, 606-609 (1994).
20. C. Kanaka-Gantenbein, E. Dicou, P. Czernichow, R. Scharfmann, Presence of nerve growth factor and its receptors in an in vitro model of islet cell development: implication in normal islet morphogenesis. Endocrinology 136, 3154-3162 (1995).
21. U. Ahlgren, S. L. Pfaff, T. M. Jessell, T. Edlund, H. Edlund, Independent requirement for ISL1 in formation of pancreatic mesenchyme and islet cells. Nature (London) 385, 257-60 (1997).
22. G. Gradwohl, A. Dierich, M. LeMeur, F. Guillemot, Hes genes regulate sequential stages of neurogenesis in the olfactory epithelium. Proc Natl Acad Sci USA 97, 1607-1611 (2000).
23. L. Bouwens, W. G. Lu, R. De Krijger, Proliferation and differentiation in the human fetal endocrine pancreas. Diabetologia 40, 398-404 (1997).
24. I. Swenne, Pancreatic beta-cell growth and diabetes mellitus. Diabetologia 35, 193-201 (1992).
25. A. Hayek, G. M. Beattie, V. Cirulli, A. D. Lopez, C. Ricordi, J. S. Rubin. Growth factor/matrix-induced proliferation of human adult beta-cells. Diabetes 44, 1458-1460 (1995).
26. I. Swenne, L. A. Borg, C. J. Crace, A. Schnell Landstrom, Persistent reduction of pancreatic beta-cell mass after a limited period of proteinenergy malnutrition in the young rat. Diabetologia 35, 939-945 (1992).
27. S. Bonner-Weir, D. Deery, J. L. Leahy, G. C. Weir. Compensatory growth of pancreatic beta-cells in adult rats after short-term glucose infusion. Diabetes 38, 49-53 (1989).
28. G. Marynissen, L. Alerts, F. A. Van Assche, J The endocrine pancreas during pregnancy and lactation in the rat. Develop. Physiol. 5, 373-381 (1983).
29. T. C. Brelje, D. W. Scharp, P. E. Lacy, Effect of homologous placental lactogens, prolactins, and growth hormones on islet B-cell division and insulin secretion in rat, mouse, and human islets: implication for placental lactogen regulation of islet function during pregnancy. Endocrinol. 132, 879 (1993).
30. L. Rosenberg and A. I. Vinik, Trophic stimulation of the ductular-islet cell axis: a new approach to the treatment of diabetes. Adv. Exp. Med Biol. 321, 95-104 (1992).

31. L. Korcakova, Mitotic division and its significance for regeneration of granulated B-cells in the islets of Langerhans in alloxan-diabetic rats. Folia. Morphol. 14, 24-30 (1971).
32. D. Cantenys, B. Portha, M. C. Dutrillaux, Histogenesis of the endocrine pancreas in newborn rats after destruction by streptozotocin. An immunocytochemical study. Virchows. Arch. Cell. Pathol. 35, 109 (1981).
33. S. Bonner-Weir, L. A. Baxter, G. T. Schuppin, F. E. Smith, Diabetes 42, 1715-1720 (1993).
34. H. Kern and J. Logothetopolous, A second pathway for regeneration of adult exocrine and endocrine pancreas. A possible recapitulation of embryonic development. Diabetes 19, 145 (1970).
35. J. Logothetopolous and E. A. Bell, Histological and autoradiographic studies of the islets of mice injected with insulin antibody. Diabetes 15, 205-211 (1966).
36. M. S. Rao, R. S. Dwivedi, A. V. Yeldani, Role of periductal and ductular epithelial cells of the adult rat pancreas in pancreatic hepatocyte lineage. A change in the differentiation commitment. Am. J. Pathol. 134, 1069 (1989).
37. D. Gu and N. Sarvetnick, Development 118, 33 (1993).
38. C. V. Weaver, R. L. Sorensen, H. C. Kuang, Immunocytochemical localization of insulin-immunoreactive cells in the pancreatic ducts of rats treated with trypsin inhibitor. Diabetologia 28, 781 (1985).
39. T. Otonlcoski, G. M. Beattie, J. S. Rubin, A. D. Lopez, A. Baird, A, Hayek Hepatocyte growth factor/scatter factor has insulinotropic activity in human fetal pancreatic cells. Diabetes 43, 947 (1994).
40. L. Bouwens and D. G. Pipeleers, Extra-insular beta cells associated with ductules are frequent in adult human pancreas. Diabetologia 41, 629 (1998).
41. I. R. Lemischka, D. H. Raulet, R. C. Mulligan, Developmental potential and dynamic behavior of hematopoietic stem cells. Cell 45, 917-927 (1986).
42. C. T. Jordan, I. R. Lemischka, Genes Dev 4, 220-232 (1990).
43. B. A. Reynolds, S. Weiss, Generation of neurons and astrocytes from isolated cells of the adult mammalian central nervous system. Science 255, 1707-1710 (1992).
44. C. B. Johansson, S. Momma, D. L. Clarke, M. Risling, U. Lendahl, J. Frisen, Identification of a neural stem cell in the adult mammalian central nervous system. Cell 96, 25-34 (1999).
45. C. S. Potten, C. Booth, D. M. Pritchard, The intestinal epithelial stem cell: the mucosal governor. Int J Exp Pathol 78, 219-243 (1997).
46. F. M. Watt, Epidermal stem cells: markers, patterning and the control of stem cell fate. Philos Trans R Soc London B 353, 831-837 (1998).
47. M. R. Alison, Liver stem cells: a two compartment system. Curr opin Cell Biol 10, 710-715 (1998).
48. T. M. Dexter, L. H. Coutinho, E. Spooncer, et al. Stromal cells in haemopoiesis. Ciba Found Symp 148, 76-86 (1990).
49. C. J. Civin, L. C. Strauss, M. J. Fackler, T. M. Trischmann, J. M. Wiley, M. R. Locken, Positive stem cell selection—basic science. Prog Clin Biol Res 333, 387-4011990.
50. C. M. Heyworth, N. G. Testa, A. M. Buckle, A. D. Whetton, In: Stem Cells, Academic Press Ltd, pp 243-441 (1997).
51. I. R. Lemischka, The power of stem cells reconsidered? Proc Natl Acad Sci USA 9: 14193-14195 (1999).
52. C. R. Bjornson, R. L. Rietze, B. A. Reynolds, C. M. Magli, and A. L. Vescovi, Turning brain into blood: a hematopoietic fate adopted by adult neural stem cells in vivo. Science 283, 534-537 (1999).
53. K. A. Jackson, T. Mi, M. A. Goodell, Hematopoietic potential of stem cells isolated from murine skeletal muscle. Proc Natl Acad Sci USA 96, 14482-14486 (1999).
54. E. Gussoni, Y. Soneoka, C. D. Strickland, E. A. Buzney, M. K. Khan, A. F. Flint, L. M. Kunkel, R. C. Mulligan. Dystrophin expression in the mdx mouse restored by stem cell transplantation. Nature (London) 401: 390-394 (1999).
55. M. R. Alison, R. Poulsom, R. Jeffery, A. P. Dhillon, A. Quaglia, J. Jacob, M. Novelli, G. Prentice, J. Williamson, N. A. Wright. Hepatocytes from non-hepatic adult stem cells. Nature (London) 406, 257 (2000).
56. M. F. Pittenger, A. M. MacKay, S. C. Beck, R. K. Jaiswal, R. Douglas, J. D. Mosca, M. A. Moorman, D. W. Simonetti, S. Craig, D. R. Marshak, Multilineage potential of adult human mesenchymal stem cells. Science 284, 143-146 (1999).
57. M. Schuldiner, O. Yanuka, J. Itskovitz-Eldor, D. A. Melton, From the cover: effects of eight growth factors on the differentiation of cells derived from human embryonic stem cells. Proc Natl Acad Sci USA 97, 11307-11312 (2000).
58. D. Woodbury, K. Reynolds, I. B. Black. Adult bone marrow stromal stem cells express germline, ectodermal, endodermal, and mesodermal genes prior to neurogenesis. J Neuro Sci 96: 908-917 (2002).
59. L. R. Zhao, W. M. Duan, M. Reyes, C. D. Keene, C. M. Verfaille, W. C. Low. Human bone marrow stem cells exhibit neural phenotypes and ameliorate neurological deficits after grafting into the ischemic brain of rats. Experimental Neurology 174: 11-20 (2002).
60. Y. Jiang, B. N. Jahagirdar, R. L. Reinhardt, R. E. Schwartz, C. D. Keene et al. Pluripotency of mesenchymal stem cells derived from adult marrow. Nature (London) 20: 1-9 (2002).
61. N. Terada, T. Hamazakit, M. Oka, M. Hoki, D. M. Mastalerz, Y. Nakano, E. M. Meyer, L. Morel, B. E. Petersen, E. W. Scott. Bone marrow cells adopt the phenotype of other cells by spontaneous fusion. Nature (London) 416: 542-545 (2002).
62. W. A. Wells. Is differentiation in trouble. J Cell Biol 157: 15-18 (2002).
63. M. F. Pittenger, A. M. MacKay, S. C. Beck, R. K. Jaiswal, R. Douglas, J. D. Mosca, M. A. Moorman, D. W. Simonetti, S. Craig, D. R. Marshak, Science 284, 143-146 (1999).
64. M. Schuldiner, O. Yanuka, J. Itskovitz-Eldor, D. A. Melton, Proc Natl Acad Sci USA 97, 11307-11312 (2000).
65. T. Dominique, T. Halvorsen, C. Dementerco, B. Tyrberg, P. I. Ansari, M. Loy et al. Beta cell differentiation from human pancreatic cell line in vitro and in vivo. Mol Endocrinol 15: 476-483 (2001).

1. A method of transdifferentiating mammalian non-pancreatic stem cells to enter the pancreatic differentiation pathway, comprising:
culturing said stem cells under conditions that permit the expression of pancreatic differentiation markers,
wherein said conditions are selected from the group consisting of:
a) culture conditions that promote cell clustering;
b) medium comprising an added factor that comprises at least one of glucagon-like peptide-1 (GLP-1), hepatocyte growth factor (HGF) and nicotinamide; and
c) conditions of both (a) and (b).

2. The method of claim 1 wherein the pancreatic markers that are expressed are selected from the group consisting of Pdx-1, Isl-1, Pax-4, Pax-6, Glut-2, glucagon, somatostatin, pancreatic peptide (PP) and insulin.

3. The method of claim 1 wherein said transdifferentiated cells respond to contact with glucose by secretion of insulin.

4. The method of claim 1 wherein said culture conditions that promote cell clustering are low binding tissue culture plates, extracellular matrix, or both.

5. The method of claim 1 wherein said medium comprises one or more components selected from the group consisting of Dulbecco's Minimal Essential Medium (DMEM) with high glucose and sodium pyruvate; bovine serum albumin (BSA); 2-mercaptoethanol; fetal calf serum (FCS); penicillin and streptomycin (Pen-Strep); insulin, transferrin and selenium (ITS); and Fungizone®.

6. The method of claim 1 wherein the added factors comprise GLP-1, HGF and nicotinamide.

7. The method of claim 6 wherein GLP-1 has a concentration of about 100 nM, HGF has a concentration of about 20 ng/ml and nicotinamide has a concentration of about 10 nM.

8. The method of claim 1 wherein said non-pancreatic stem cell is a mesenchymal stem cell (MSC).

9. The method of claim 8 wherein the MSC is CD105+, CD166+, CD29+ and CD44+.

10. The method of claim 1 wherein the stem cells are human.

11. A method of producing an endocrine hormone comprising the method of claim 1, and further comprising the step of continuing to culture said transdifferentiated cells in said medium, whereby an endocrine hormone may be produced.

12. A method of treating a mammal with a pancreatic disorder, comprising:
a) culturing non-pancreatic stem cells according to claim 1, whereby said stem cells transdifferentiate to the pancreatic differentiation pathway;
b) using a product of the culture of step (a) to treat said mammal.

13. The method of claim 12 wherein the pancreatic disorder is an insulin-requiring disorder.

14. The method of claim 12 wherein the product in step (b) is an endocrine hormone that is administered to the mammal.

15. The method of claim 12 wherein the product in step (b) is transdifferentiated cells, and said method further comprises:
c) implanting said product into said mammal.

16. The method of claim 15 wherein said implantation is in pancreatic, kidney or liver tissue, or in a subcutaneous pocket.

17. The method of claim 12 wherein said transdifferentiated stem cells are transdifferentiated MSCs.

18. The method of claim 12 wherein the non-pancreatic stem cells originate from an individual that is the same as the treated mammal of step (b).

19. The method of claim 15 wherein said implanted transdifferentiated cells are encapsulated in an endocrine hormone permeable capsule.

20. The method of claim 19 wherein said implanted, encapsulated, transdifferentiated cells are autologous to the mammal.

21. The method of claim 19 wherein said implanted, encapsulated, transdifferentiated cells are allogeneic to the mammal.

22. The transdifferentiated MSC produced by the method of claim 8.

23. A transdifferentiated MSC that expresses mRNA for Isl-1, Pax-6, PP, insulin, glucagon and somatostatin.

24. A transdifferentiated cell produced by the method of claim 1.

25. A therapeutic composition comprising a transdifferentiated MSC encapsulated in an endocrine hormone permeable capsule.
